(19) United States
(12) Patent Application Publication
(10) Pub. No.: US 2021/0251799 A1
(43) Pub. Date: Aug. 19, 2021

(54) MALE GENITAL DISCHARGE ABSORPTION AND RETENTION DEVICE

(71) Applicant: Michael Taylor, Inglewood, CA (US)

(72) Inventor: Michael Taylor, Inglewood, CA (US)

(21) Appl. No.: 16/792,811

(22) Filed: Feb. 17, 2020

(51) Int. Cl. A61F 5/453; A61F 5/441; A61F 13/471; A61F 13/15

(52) U.S. Cl. CPC A61F 5/453; A61F 13/15268; A61F 13/471; A61F 5/441

(57) ABSTRACT

A male genital discharge absorption and retention device is disclosed herein. The purpose of the device is to absorb any discharge from the male meatus, which may also be known as the external urethral orifice. The device aids in the prevention of spotting or staining on the clothing of a user wearing the device and affected by penile discharge. A fabric or other appropriate material is fitted over the glans penis and penile corona and may be held in place by an appropriate attachment mechanism. The anatomy of the male genitalia also aids in securing the male genital discharge absorption and retention device to the penis. Within the device, positioned against the meatus of the male genitalia, may be a thicker absorption mechanism intended to soak up any penile discharge. The device, or any component thereof, may further comprise an antibacterial material of constriction or an antibacterial layer of material.

### TECHNICAL FIELD OF THE INVENTION

The present invention relates in general to wearable medical devices, and, more specifically, to a male genital discharge absorption and retention device.

### COPYRIGHT AND TRADEMARK NOTICE

A portion of the disclosure of this patent application may contain material that is subject to copyright protection. The owner has no objection to the facsimile reproduction by anyone of the patent document or the patent disclosure, as it appears in the Patent and Trademark Office patent file or records, but otherwise reserves all copyrights whatsoever.

Certain marks referenced herein may be common law or registered trademarks of third parties affiliated or unaffiliated with the applicant or the assignee. Use of these marks is by way of example and should not be construed as descriptive or to limit the scope of this invention to material associated only with such marks.

### BACKGROUND OF THE INVENTION

The external male genitalia are very generally comprised of the scrotum and the penis. In normal anatomy the scrotum houses a pair of testicles, each having a single epididymis, and a pair of ductus deferentia connecting these structures to the penis. Sperm are produced in the testicles and mature as they travel through the ductus deferentia to the seminal vesicles. Secretions from the male prostate gland are then combined with the sperm to produce semen, which is delivered through the urethra within the penis for normal discharge during ejaculation.

The male bladder is contained within the lower abdomen and comprises a muscular sac meant to store urine produced in the kidneys. Urine enters the bladder via a pair of ureters, and passes out via a single urethra that, then, passes through the prostate gland into the shaft of the penis to allow for normal discharge during urination.

Penile discharge, which may be any unintended or pathologic discharge from the urethra of the penis, may be watery or cloudy. A watery discharge is generally clear and usually indicates a non-infectious cause of the discharge. A cloudy discharge, which can be white, yellow, green, or any other color, usually indicates that the discharge contains pus, suggesting an infectious origin.

Watery discharge, specifically, can result from such innocuous causes as failing to properly clear the penis after urinating, which may result in dribbling, or from pre-ejaculation caused by sexual arousal. Other causes of watery discharge may include inflammation of any component of the urinary or seminal systems, such urethritis, prostatitis, or epididymitis.

Cloudy discharge, on the other hand, may result from any infectious inflammation or direct infection of the various components of the urinary or seminal systems. Viral or bacterial bladder and prostate infections may result in the depositing of pus within the urine or semen, as can sexually transmitted infections such as chlamydia, gonorrhea, herpes, and other common pathologies.

Such penile discharge, whether of infectious or non-infectious origin, may cause spotting or staining of clothing worn by the affected person. Some solutions exist to prevent such spotting or staining, especially in the form of child and adult diapers. Diapers, though, may cause embarrassment to the wearer by virtue of their bulk or by social stigma against the user. Diapers, also, may not be easily carried, such as when there is the need for additional diapers if the worn item is soiled, and may not be easily changed without the user also removing the article of clothing worn over the diaper.

Thus, there is a need in the art for a male genital discharge absorption and retention device that comprises a small form factor while also providing effective penile discharge absorption to prevent spotting and staining of the wearer's clothing. The male genital discharge absorption and retention device may further be easily carried and easily changed without the need for additional carrying devices or the removal of articles of clothing worn over the device. It is to these ends that the present invention has been developed.

### BRIEF SUMMARY OF THE INVENTION

To minimize the limitations in the prior art, and to minimize other limitations that will be apparent upon reading and understanding the present specification, the present invention describes a male genital discharge absorption and retention device.

It is an objective of the present invention to provide a male genital discharge absorption and retention device that may comprise a wearable device.

It is another objective of the present invention to provide a male genital discharge absorption and retention device that may comprise a fabric main body.

It is another objective of the present invention to provide a male genital discharge absorption and retention device that may comprise an absorbent main body.

It is another objective of the present invention to provide a male genital discharge absorption and retention device that may comprise an attachment mechanism.

It is another objective of the present invention to provide a male genital discharge absorption and retention device that may comprise an absorption mechanism.

It is another objective of the present invention to provide a male genital discharge absorption and retention device that may be worn on the male genitalia of a user.

It is another objective of the present invention to provide a male genital discharge absorption and retention device that may absorb discharge secreted from the male genitalia of a user.

It is another objective of the present invention to provide a male genital discharge absorption and retention device that may comprise a single-component construction.

It is another objective of the present invention to provide a male genital discharge absorption and retention device that may comprise a multiple-component construction.

It is another objective of the present invention to provide a male genital discharge absorption and retention device that may comprise a resilient material of construction.

It is another objective of the present invention to provide a male genital discharge absorption and retention device that may comprise a cleanable material of construction.

It is another objective of the present invention to provide a male genital discharge absorption and retention device that may comprise a reusable material of construction.

It is another objective of the present invention to provide a male genital discharge absorption and retention device that may comprise an antimicrobial layer.

It is another objective of the present invention to provide a male genital discharge absorption and retention device that may comprise an antimicrobial material of construction.

It is another objective of the present invention to provide a male genital discharge absorption and retention device that may comprise an analgesic layer.

It is another objective of the present invention to provide a male genital discharge absorption and retention device that may comprise an analgesic material of construction.

These and other advantages and features of the present invention are described herein with specificity so as to make the present invention understandable to one of ordinary skill in the art, both with respect to how to practice the present invention and how to make the present invention.

### BRIEF DESCRIPTION OF THE SEVERAL VIEWS OF THE DRAWINGS

Elements in the figures have not necessarily been drawn to scale in order to enhance their clarity and improve understanding of these various elements and embodiments of the invention. Furthermore, elements that are known to be common and well understood to those in the industry are not depicted in order to provide a clear view of the various embodiments of the invention.

FIG. 1 illustrates an upper isometric perspective view of a male genital discharge absorption and retention device, as contemplated by the present disclosure;

FIG. 2 illustrates a lower isometric perspective view of a male genital discharge absorption and retention device, as contemplated by the present disclosure;

FIG. 3 illustrates a side elevation view of a male genital discharge absorption and retention device, as contemplated by the present disclosure;

FIG. 4 illustrates a bottom perspective view of a male genital discharge absorption and retention device, as contemplated by the present disclosure;

FIG. 5 illustrates an upper isometric perspective view of a male genital discharge absorption and retention device with the enclosure component pulled back exposing the attachment mechanism, as contemplated by the present disclosure;

FIG. 6 illustrates the attachment mechanism of a male genital discharge absorption and retention device, as contemplated by the present disclosure;

FIG. 7 illustrates the absorption mechanism of a male genital discharge absorption and retention device, as contemplated by the present disclosure;

FIG. 8 illustrates an exploded view of the various components of a male genital discharge absorption and retention device, as contemplated by the present disclosure;

FIG. 9 illustrates the relative orientation of a male genital discharge absorption and retention device over an exemplary male genital, as contemplated by the present disclosure; and

FIG. 10 illustrates a male genital discharge absorption and retention device worn on an exemplary male genital.

### DETAILED DESCRIPTION OF THE INVENTION

Certain terminology is used in the following description for reference only and is not limiting. The words “front,” “rear,” “anterior,” “posterior,” “lateral,” “medial,” “upper,” “lower,” “outer,” “inner,” and “interior” refer to directions toward and away from, respectively, the geometric center of the invention, and designated parts thereof, in accordance with the present disclosure. Unless specifically set forth herein, the terms “a,” “an,” and “the” are not limited to one element, but instead should be read as meaning “at least one.” The terminology includes the words noted above, derivatives thereof, and words of similar import.

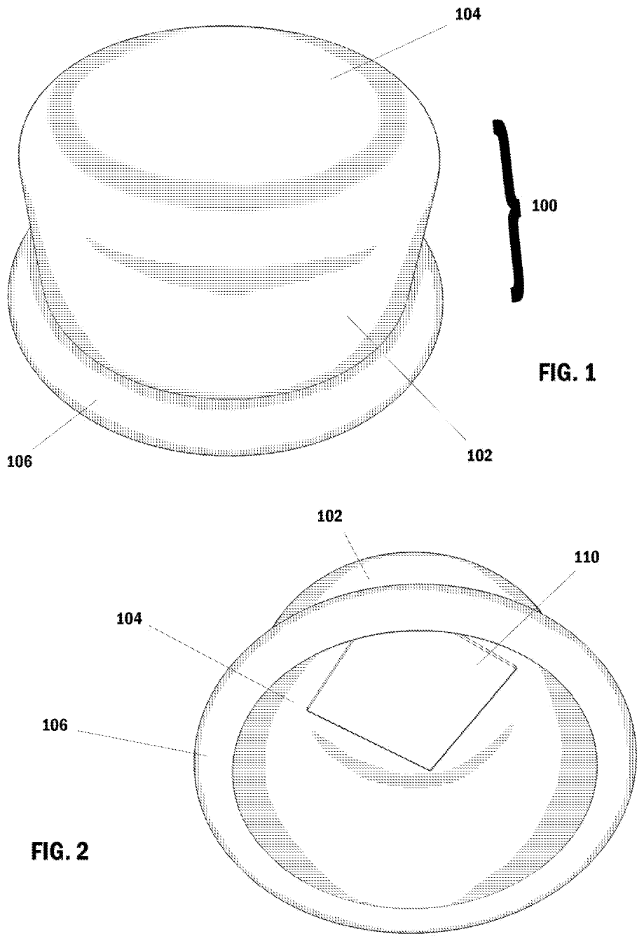

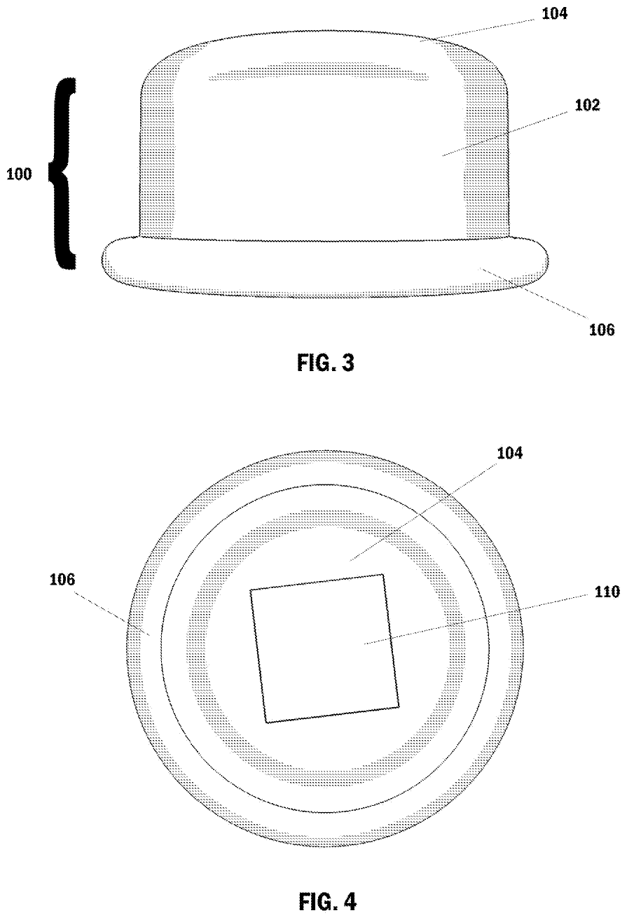

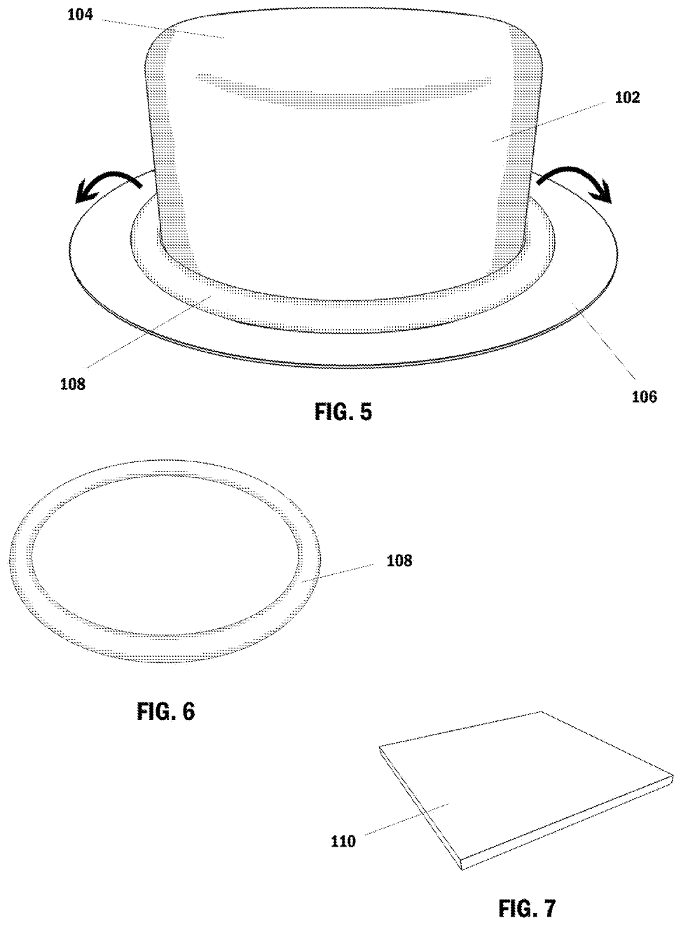

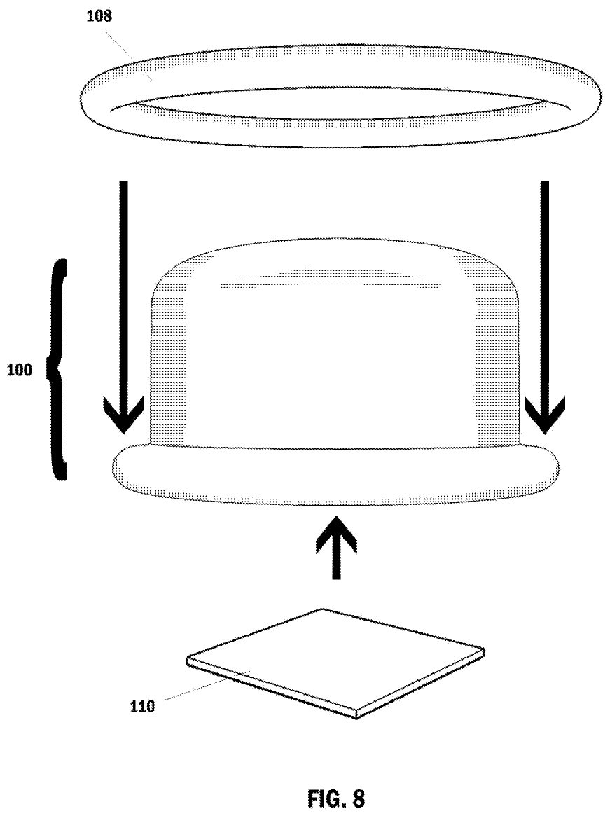

The illustrations of FIGS. 1-10 illustrate a male genital discharge absorption and retention device, as contemplated by the present disclosure. The purpose of the male genital discharge absorption and retention device is to absorb any discharge from the male meatus, which may also be known as the external urethral orifice. The male genital discharge absorption and retention device aids in the prevention of spotting or staining on the clothing of a user wearing the device and affected by penile discharge. A fabric or other appropriate material is fitted over the glans penis and penile corona and may be held in place by an appropriate attachment mechanism. The anatomy of the male genitalia also aids in securing the male genital discharge absorption and retention device to the penis. Within the male genital discharge absorption and retention device, positioned against the meatus of the male genitalia, may be a thicker absorption mechanism intended to soak up any penile discharge. The male genital discharge absorption and retention device, or any component thereof, may further comprise an antibacterial material of constriction or an antibacterial layer of material.

The illustrations of FIGS. 1-8 illustrate a male genital discharge absorption and retention device, as contemplated by the present disclosure. The device may comprise, generally, a main body 100 having a vertical component 102, a horizontal component 104, and an enclosure component 106. The main body 100 may be designed so as to be highly flexible and formable while substantially maintaining its general shape. The various components of the main body 100 may comprise a single-component construction or may comprise a multiple-component construction. In an embodiment wherein the main body 100 comprises a multiple-component construction the various components may be attached to one another by any appropriate means such as, for example, by gluing, welding, or stitching.

In an embodiment wherein the main body 100 comprises a multiple-component construction the vertical component 102 of the main body 100 may substantially comprise a planar sheet of material formed into a substantially cylindrical shape, and may further comprise an upper edge, a lower edge, an outer side, and an inner side. The horizontal component 104 of the main body 100 may substantially comprise a circular planar sheet, and may further comprise a perimeter, an upper side, and a lower side. The enclosure component 106 may substantially comprise a planar sheet of material formed into a substantially cylindrical shape, and may further comprise an upper edge, a lower edge, an outer side, and an inner side.

In one embodiment the perimeter of the horizontal component 104 may be attached to the upper edge of the vertical component 102 so as to form a shape having a semi-circular or u-shaped cross-section. The lower edge of the vertical component 102 may be attached to the upper edge of the enclosure component 106 so as to form a generally continuous cylindrical plane when both components are fully straightened. The lower edge of the enclosure component 106 may, then, be curved around and attached to an area on the outer surface of the vertical component near the lower edge of the vertical component 102 so as to form a loop around the perimeter of the main body 100.

In an embodiment wherein the main body 100 comprises a single-component construction the horizontal component, vertical component, and enclosure component may comprise a single sheet of material appropriately shaped to create the structure and orientation described in the multiple-component design.

In some embodiments the vertical component 102, horizontal component 104, and enclosure component 106 of the main body 100 of the male genital discharge absorption and retention device disclosed herein may be substantially constructed of any suitable material or combination of materials, but typically is constructed of a resilient material or combination of materials such that the combination is resistant to tearing and damage as a result of compression, flexion, or submersion in water. As an example, and without limiting the scope of the present invention, various exemplary embodiments of the combination may be substantially constructed of one or more materials of fabric, latex, nitrile, rubber, polyurethane, animal skin, plastic, or combinations thereof.

The male genital discharge absorption and retention device may further comprise an attachment mechanism 108, which may comprise a substantially circular or oval shape having a perimeter and a central opening. The attachment mechanism 108 may be designed so as to be somewhat flexible and formable while substantially maintaining its general shape. The attachment mechanism 108 may be installed within the loop of the enclosure component 106 and may provide structural support to the vertical component 102 and horizontal component 104 of the main body 100. The perimeter of the attachment mechanism 108 may be large enough to fit comfortably around a head or a shaft of a penis, and the material of construction of the attachment mechanism 108 may be partially flexible to permit installation, wearing, and removal of the main body 100 without causing discomfort to a user of the device.

The main body 100 may be sized large enough that the attachment mechanism 108 is placed proximal to a corona of the penis, the base of the glans penis usually forming a projection resulting in an area of slightly wider diameter, such that the corona of the penis aids in securing the main body 100 to a user of the device.

In some embodiments the attachment mechanism 108 of the male genital discharge absorption and retention device disclosed herein may be substantially constructed of any suitable material or combination of materials, but typically is constructed of a resilient material or combination of materials such that the combination is resistant to tearing and damage as a result of compression, flexion, or submersion in water. As an example, and without limiting the scope of the present invention, various exemplary embodiments of the combination may be substantially constructed of one or more materials of fabric, latex, rubber, polyurethane, animal skin, plastic, or combinations thereof.

The male genital discharge absorption and retention device may further comprise an absorption mechanism 110, which may comprise any appropriately shaped sheet of absorbent material having an upper side, a lower side, and a perimeter. The absorption mechanism 110 may be designed so as to be somewhat flexible and formable while substantially maintaining its general shape. The upper side of the absorption mechanism 110 may be fixedly attached to the lower side of the horizontal component 104 of the main body 100 by any appropriate means such that the lower side of the absorption component 110 is positioned near or against the meatus of a user of the device.

The shape and size of the absorption mechanism 110 may vary as desired, and the absorption mechanism 110 may partially or completely cover the lower side of the horizontal component 104. In one embodiment the absorption mechanism 110 may further partially or completely cover the inner side of the vertical component 102.

In some embodiments the absorption mechanism 110 of the male genital discharge absorption and retention device disclosed herein may be substantially constructed of any suitable material or combination of materials, but typically is constructed of an absorbent material or combination of materials such that the combination is resistant to damage as a result of exposure to fluids. As an example, and without limiting the scope of the present invention, various exemplary embodiments of the combination may be substantially constructed of one or more materials of fabric, polypropylene, cellulose, wood pulp, hemp fiber, animal skin, or combinations thereof.

In some embodiments the male genital discharge absorption and retention device may further comprise a material of construction that either comprises a material having antimicrobial properties or comprises a layering of antimicrobial material or coating. Antimicrobial properties comprise the characteristic of being antibacterial, biocidal, microbicidal, anti-fungal, anti-viral, or other similar characteristics.

In some embodiments the male genital discharge absorption and retention device may further comprise a material of construction that either comprises a material having analgesic properties or comprises a layering of analgesic material or coating. Analgesic properties comprise the characteristic of relieving pain, inflammation, itching, discomfort, or other similar characteristics.

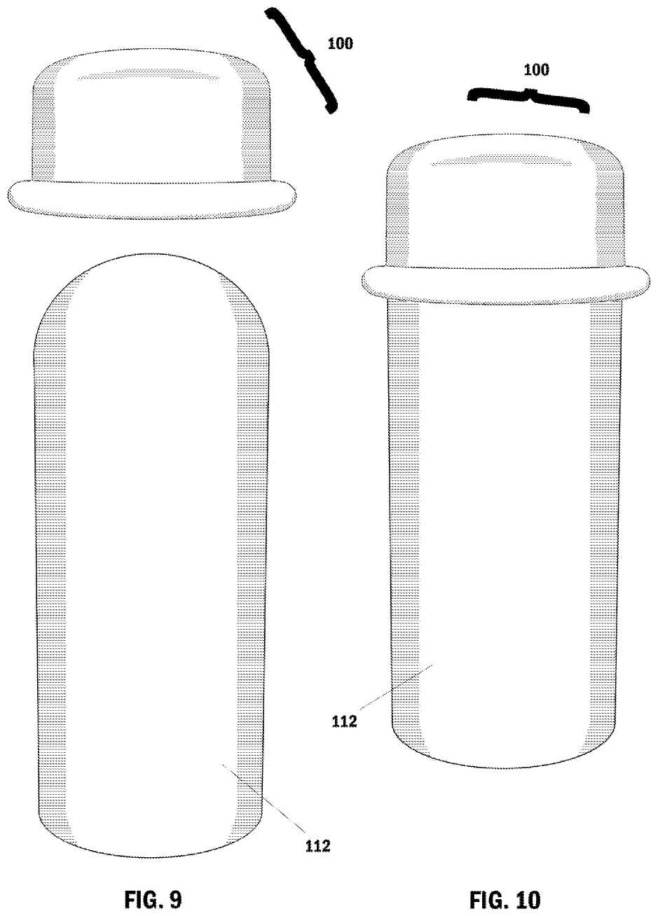

The illustrations of FIGS. 9 and 10 illustrate the installation and wearing of a male genital discharge absorption and retention device, as contemplated by the present disclosure. To begin using the device a user may position the main body 100 over an exemplary penis 112 such that a central opening of the enclosure compartment 106 formed by the attachment mechanism 108 is facing the tip of the user's penis 112. The user may then advance the tip of the penis 112 through the central opening until the tip of the penis 112 is placed near or against the absorption mechanism 110 or the attachment mechanism 108 passes over the corona of the penis 112. The attachment mechanism 108 may attach to the shaft of the penis 112 by friction and, thus, may retain the absorption mechanism 110 in front of, against, or around the meatus of the penis 112.

Removal of the male genital discharge absorption and retention device involves detaching the attachment mechanism 108 from the shaft of the penis 112 and withdrawing the penis 112 from the main body 100. Due to the size and form factor of the device a user may install, wear, remove, and replace the male genital discharge absorption and retention device without removing any article of clothing worn over the device.

While the invention has been described in connection with what is presently considered to be the most practical and preferred embodiments, it is to be understood that the invention is not to be limited to the disclosed embodiments, but, on the contrary, is intended to cover various modifications and equivalent arrangements included within the spirit and scope of the appended claims.

I claim:

1. A male genital discharge absorption and retention device, comprising:
a main body;
an attachment mechanism; and
an absorption mechanism;
wherein said main body further comprises a vertical component, a horizontal component, and an enclosure component;
wherein said vertical component further comprises an upper edge, a lower edge, an outer side, and an inner side;
wherein said horizontal component further comprises a perimeter, an upper side, and a lower side;
wherein said enclosure component further comprises an upper edge and a lower edge;
wherein said upper edge of said vertical component is attached to said perimeter of said horizontal component;
wherein said lower edge of said vertical component is attached to said upper edge of said enclosure component;
wherein said lower edge of said enclosure component is attached to an area on said outer side of said vertical component to form a loop;
wherein said attachment mechanism is installed within said loop; and
wherein said absorption mechanism is attached to said lower side of said horizontal component.

2. The invention of claim 1,
wherein said attachment mechanism comprises a ring shape.

3. The invention of claim 2,
wherein said absorption mechanism is also attached to said inner side of said vertical component.

4. The invention of claim 3,
wherein said ring shape induces a shape in said main body having a substantially u-shaped cross-section.

5. The invention of claim 3, further comprising:
a single-component construction.

6. The invention of claim 3, further comprising:
a multiple-component construction.

7. The invention of claim 3, further comprising:
a resilient material of construction.

8. The invention of claim 3, further comprising:
a cleanable material of construction.

9. The invention of claim 3, further comprising:
a reusable material of construction.

10. The invention of claim 3, further comprising:
an antimicrobial layer.

11. The invention of claim 3, further comprising:
an antimicrobial material of construction.

12. The invention of claim 3, further comprising:
an analgesic layer.

13. The invention of claim 3, further comprising:
an analgesic material of construction.
